(19) United States
(12) Patent Application Publication
(10) Pub. No.: US 2007/0179343 A1
(43) Pub. Date: Aug. 2, 2007

(54) Suction retraction surgical instrument

(76) Inventor: Alexis Paul Shelokov, Dallas, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(21) Appl. No.: 11/342,510

(22) Filed: Jan. 30, 2006

(51) Int. Cl. A61B 1/32

(52) U.S. Cl. 600/205; 600/210

(57) ABSTRACT

A suction retraction surgical instrument retracts tissue and removes smoke, fluid, debris and other matter from a surgical site. The instrument includes an elongate body having a proximal section, a distal section and a central section. A vacuum or suction channel is defined within the elongate body. The instrument includes a retractor at the distal section of the elongate body. The retractor has an inner surface and an outer surface defining a suction port. The suction port is adapted to remove smoke, blood and other matter from the surgical site. The instrument includes a suction connector at the proximal section of the elongate body. The suction connector is in communication with the vacuum channel. The suction connector is adapted to aid in communication with a vacuum system for removal of smoke, fluid and other matter from the surgical site.

This invention relates to surgical instruments and, more particularly, to a suction retraction surgical instrument for retracting tissue and for removing matter from a surgical site.

### BACKGROUND OF THE INVENTION

In spinal and other surgery, the tissue is divided from the bone using a Bovie cautery or an electric knife. As the tissue is divided from the bone, the tissue is retracted. At present, a Cobb elevator is generally used by surgeons to retract the tissue.

The Cobb elevator consists of a smooth blade at the tip of an elongated handle. The blade is used to displace the tissue laterally. While some surgeons scrape muscle from the bone with the Cobb elevator, most simply retract the muscle using the Cobb elevator and use the Bovie cautery to divide the muscle from the bone.

While the Cobb elevator is currently being used to retract the tissue, it is not particularly adapted for that purpose. There are several disadvantages associated with using the Cobb elevator to retract the tissue. The Cobb elevator is not adapted to firmly grasp the tissue and has a tendency to slip due to the smooth nature of the blade. The smooth nature of the blade makes it difficult to safely hold on to the tissue.

Furthermore, the Bovie cautery (or other electric knife) generates a significant amount of smoke and fluid (e.g., blood) at the surgical site. The smoke is generated by superheating of the water in the tissue and some charring also occurs in denser tissue creating debris. The accumulation of smoke and fluid obstructs the vision of the operating surgeon. In order to improve the vision of the operating surgeon, the smoke and the fluid is removed from the site. A separate suction device is usually held by an assistant to remove the smoke and the fluid from the site. In a limited space (i.e., the surgical site), the presence of two instruments complicates the procedure and makes the manipulation of the instruments difficult.

Accordingly, there exists a need for a surgical instrument that can safely retract tissue during a surgery. Also, there exists a need for a surgical instrument that can remove the smoke and the fluid from the surgical site without impeding the Bovie cautery.

### BRIEF SUMMARY OF THE INVENTION

A suction retraction surgical instrument retracts tissue and removes smoke, fluid, debris and other matter from a surgical site. The instrument includes an elongate body having a proximal section, a distal section and a central section. A vacuum or suction channel is defined within the elongate body.

The instrument includes a retractor at the distal section of the elongate body. The retractor has an inner surface and an outer surface defining a suction port. The suction port is in communication with the channel. At least a portion of the suction port includes teeth configured to retract the tissue. The suction port is adapted to remove smoke, blood and other matter from the surgical site.

The instrument includes a suction connector at the proximal section of the elongate body. The suction connector is in communication with the vacuum channel. The suction connector is adapted to aid in communication with a vacuum system for removal of smoke, fluid and other matter from the surgical site. In one embodiment, the inner surface of the retractor is substantially concave and the outer surface of the retractor is substantially convex. The central section includes an enlarged region for easy manipulation of the instrument.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1A illustrates one embodiment of a suction retraction surgical instrument.

FIG. 1B shows a suction retraction surgical instrument with a vacuum or suction channel defined inside the instrument.

FIG. 2A is a front view of a retractor.

FIG. 2B is a perspective view of the retractor with sections removed to expose the channel defined inside the instrument.

FIG. 3 illustrates a use of the instrument in a surgery.

### DETAILED DESCRIPTION OF THE INVENTION

The various features and methods of the suction retraction surgical instrument will now be described. Those skilled in the art will recognize that the instrument can be used in spinal and other types of surgery.

Throughout the description, implementation-specific details will be given on how the instrument is used. These details are provided to illustrate the preferred embodiments of the invention and not to limit the scope of the invention. The scope of the invention is set in the claims section.

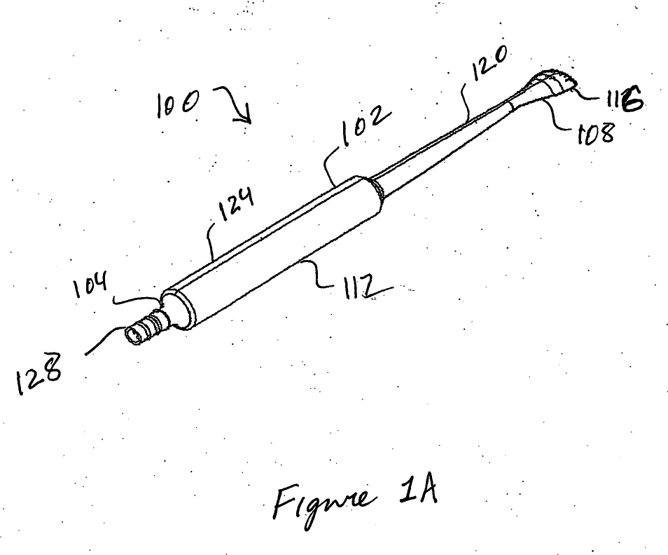

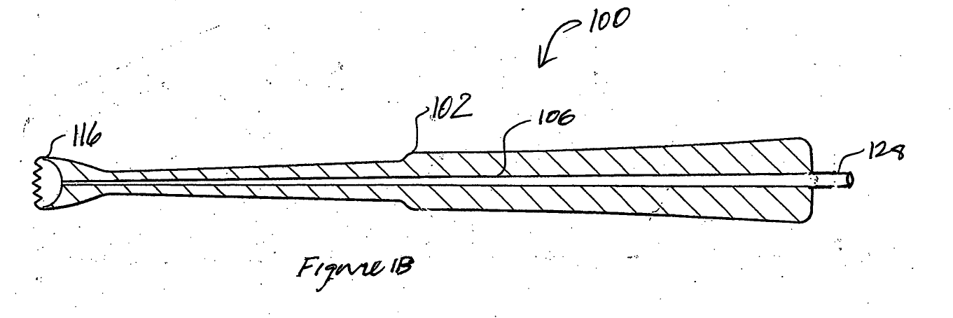

FIG. 1A illustrates one embodiment of a suction retraction surgical instrument 100. The surgical instrument 100 includes an elongate body 102 having a proximal section 104, a distal section 108 and a central section 112. A vacuum or suction channel 106 shown in FIG. 1B is defined within the elongate body 102. The channel 106 provides a path for smoke, fluid (e.g., blood) and other matter (e.g., debris) to be removed from a surgical site.

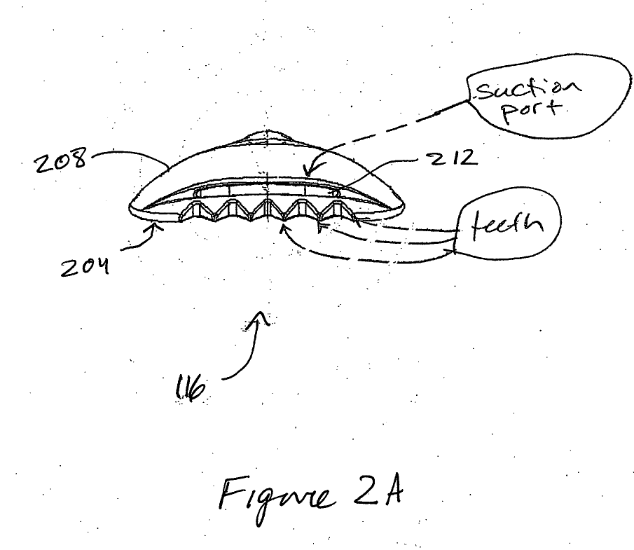

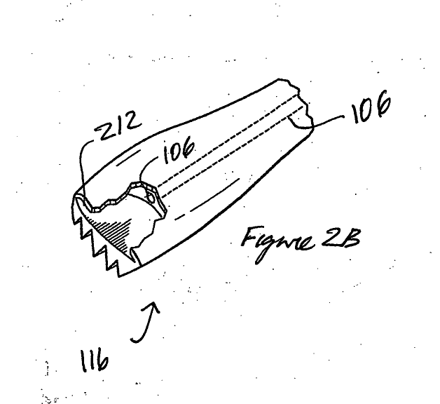

A curved retractor 116 is located at the distal section 108. FIG. 2A is a front view of the curved retractor 116. The retractor 116 includes an inner surface 204 and an outer surface 208. In one embodiment, the inner surface 204 is concave and the outer surface 208 is convex. The inner surface 204 and the outer surface 208 defines a suction port 212. In one embodiment, the suction port 212 is located at the tip of the retractor 116. The suction port 212 communicates with the channel 106 (shown in FIG. 1B) within the elongate body 202. FIG. 2B is a perspective view of the retractor 116 with sections removed to expose the channel 106 defined inside the instrument 100. As shown in FIG. 2B, the channel 106 is in communication with the suction port 212.

The retractor 116 is not sharp to have a cutting action on the tissue but rather allows the tissue to be grasped, held and retracted. In one embodiment, at least a portion of the suction port 212 includes teeth, which allows the tissue to be grasped and retracted. In one embodiment, the front-end of the inner surface 204 includes teeth. The teeth prevents the retractor from slipping as the tissue is grasped and retracted, thus allowing a safe and stable means to retract the tissue. The retractor 116 can have other shapes or forms that will allow the instrument to grasp and retract the tissue.

A shank 120 (shown in FIG. 1A) serves as a connector between the retractor 116 and the central section 112 and provides a means for forming the walls of the vacuum or suction channel 106 therethrough. A handle portion 124 in the central section 112 is slightly enlarged to provide an external configuration adapted for manual manipulation. In one embodiment, the handle portion has external elongate ridges (not shown in FIG. 1) to aid in manipulation.

A vacuum or suction connector 128 (shown in FIGS. 1A and 1B) is at the proximal end of the instrument. The connector 128 is in communication with the suction port 212 through the channel. The connector 128 is adapted to be mated to a vacuum or suction system. Smoke, fluid and debris are removed by the suction port 212 through the channel 106 and out of the connector 128.

The instrument 100 can be formed of a plastic or other suitable material. A disposable version of the instrument 100 can be made using a suitable material. The disposable version of the instrument 100 will allow the surgeons to use the instrument 100 and then dispose the instrument 100 after its use. In one embodiment, the instrument 100 can be formed from surgical steel.

In one embodiment, the instrument is formed from radio lucent material. In one embodiment, the retractor 116 is flexible. The instrument 100 may be of a suitable length (e.g., 8 inches, 10 inches, 12 inches, 14 inches, etc).

The instrument 100 has unique features that make it particularly suited for retracting tissue. The retractor 116 includes multiple serrated angulated teeth that are suitable for retracting. The suction port 212 built into the instrument 100 prevents obstruction of the operating surgeon's view. In one embodiment, the retractor 116 is slightly angulated to provide a superior and safer attack angle to retract tissue.

The instrument 100 is well suited in minimally invasive spinal surgery. It is very difficult to make a small incision and use both the Cobb elevator and a suction device when bleeding is encountered. The instrument 100 allows a surgeon to use only one instrument in a smaller wound with greater efficiency.

In operation, the instrument 100 is connected to a vacuum pump (not shown), as commonly used in surgical practice, by means of an elastic tube releasably and functionally communicating with the suction connector 128. In use, the instrument 100 is inserted through an incision, and is used to retract tissue as described before. As blood or other body and tissue fluids collect in or about the surgical site, they are evacuated through the suction port 212 and through the channel 106 into a collector of the vacuum system.

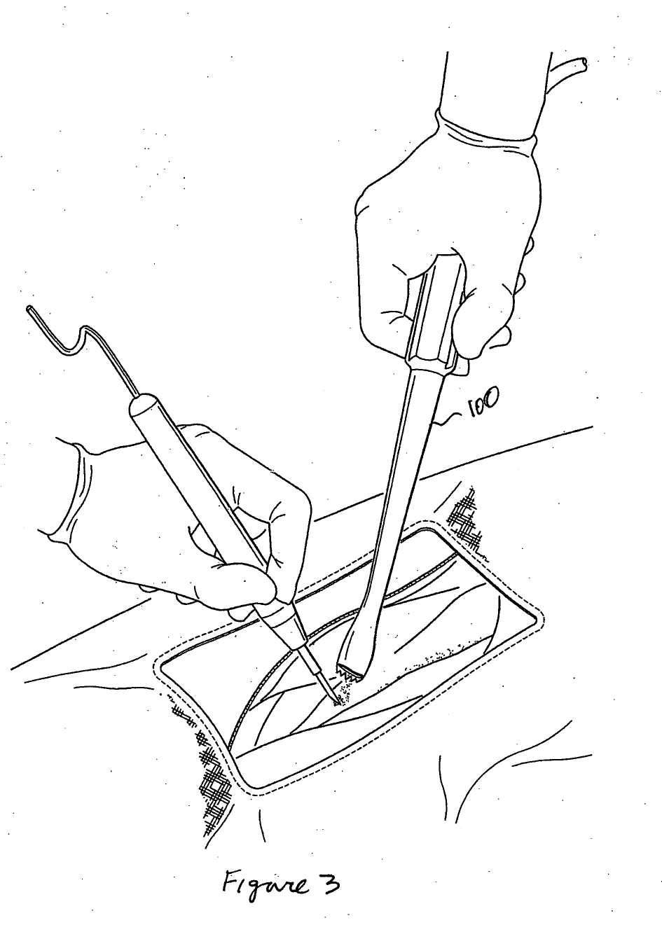

FIG. 3 illustrates the use of the instrument 100 in a surgery. As shown in FIG. 3, the instrument 100 is used to retract tissue as a Bovie cautery cuts the tissue.

Certain modifications and improvements will occur to those skilled in the art upon a reading of the foregoing description. It should be understood that all such modifications and improvements have not been described herein for the sake of conciseness and readability but are properly within the scope of the following claims.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A surgical instrument for retracting tissue and for removing matter from a surgical site during a surgery, comprising:
an elongate body including a proximal section, a distal section and a central section;
a suction channel defined within the elongate body, the suction channel providing a passage for matter to be removed from the surgical site;
a curved retractor at the distal section of the elongate body, the retractor having an inner surface and an outer surface defining a suction port, the suction port being in communication with the channel, at least a portion of the suction port including teeth configured to retract the tissue; the suction port adapted to remove matter from the surgical site during surgery; and
a suction connector at the proximal section of the elongate body, the suction connector being in communication with the channel and adapted to aid in communication with a vacuum system for removal of matter from the surgical site during the surgery.

2. The surgical instrument of claim 1, wherein the inner surface of the retractor is concave.

3. The surgical instrument of claim 1, wherein the outer surface of the retractor is convex.

4. The surgical instrument of claim 1, wherein the central section includes an enlarged region for easy manipulation of the instrument.

5. The surgical instrument of claim 1, wherein the retractor communicates with the central section by a narrow shank region.

6. The surgical instrument of claim 1, wherein the central section includes external elongate ridges to aid in the manipulation of the instrument.

7. The surgical instrument of claim 1, wherein the suction port is located at the tip of the retractor.

8. A surgical instrument for retracting tissue and for removing matter from a surgical site during a surgery, comprising:
an elongate body including a proximal section, a distal section and a central section;
a suction channel defined within the elongate body, the suction channel providing a passage for matter to be removed from the surgical site;
a retractor at the distal section of the elongate body configured to retract tissue, the retractor having an inner surface and an outer surface defining a suction port at the tip of the retractor, the suction port being in communication with the channel, the suction port adapted to remove matter from the surgical site during the surgery; and
a suction connector at the proximal section of the elongate body, the suction connector being in communication with the channel and adapted to aid in communication with a vacuum system for removal of matter from the surgical site during the surgery.

9. The surgical instrument of claim 9, wherein the inner surface of the retractor is concave.

10. The surgical instrument of claim 9, wherein the outer surface of the retractor is convex.

11. The surgical instrument of claim 9, wherein at least a portion of the suction port includes teeth adapted to retract tissue during surgery.

12. The surgical instrument of claim 9, wherein the retractor is flexible.

13. A method for retracting tissue and removing matter during a surgery using a suction retraction surgical instrument, comprising the steps of:
attaching the surgical instrument to a vacuum system;
making an incision in a selected site in a body;
inserting the surgical instrument through the insertion;
retracting the tissue the using the surgical instrument; and
removing matter from the selected site using the vacuum system,
wherein the surgical instrument comprises:
an elongate body including a proximal section, a distal section and a central section;
a suction channel defined within the elongate body, the suction channel providing a passage for matter to be removed from the surgical site;
a retractor at the distal section of the elongate body, the retractor having an inner surface and an outer surface defining a suction port, the suction port being in communication with the channel, at least a portion of the suction port including teeth configured to retract tissue; the suction port adapted to remove matter from the selected site during surgery; and
a suction connector at the proximal section of the elongate body, the suction connector being in communication with the channel and adapted to aid in communication with a vacuum system for removal of matter from the surgical site during the surgery.

14. The method of claim 14, wherein the inner surface of the retractor is substantially concave.

15. The method of claim 14, wherein the inner surface of the retractor is substantially convex.

16. A disposable surgical retraction instrument configured to retract tissue and remove matter from a surgical site during a surgery, comprising:
an elongate body including a proximal section, a distal section and a central section;
a suction channel defined within the elongate body, the suction channel providing a passage for matter to be removed from the surgical site;
a retractor at the distal section of the elongate body, the retractor having an inner surface and an outer surface defining a suction port, the suction port being in communication with the suction channel, at least a portion of the suction port including teeth configured to retract the tissue; the suction port adapted to remove matter from the surgical site during surgery; and
a suction connector at the proximal section of the elongate body, the suction connector being in communication with the suction channel and adapted to aid in communication with a vacuum system for removal of matter from the surgical site during the surgery.

17. The method of claim 16, wherein the inner surface of the retractor is concave.

18. The method of claim 16, wherein the outer surface of the retractor is convex.
